(19) United States
(12) Patent Application Publication
(10) Pub. No.: US 2015/0185227 A1
(43) Pub. Date: Jul. 2, 2015

(54) MARKER FOR PRETERM LABOUR

(71) Applicant: KING'S COLLEGE LONDON, London (GB)

(72) Inventor: Rachel M. Tribe, London (GB)

(21) Appl. No.: 14/379,794

(22) PCT Filed: Feb. 22, 2013
(86) PCT No.: PCT/GB2013/050445

§ 371 Date: Aug. 20, 2014

(30) Foreign Application Priority Data
Feb. 23, 2012 (GB) 1203217.3

(51) Int. Cl. G01N 33/68; A61K 31/57

(52) U.S. Cl. CPC G01N 33/689; A61K 31/57; G01N 2333/811; G01N 2800/368

(57) ABSTRACT

Some embodiments relate to a method of determining a risk of preterm labour in a subject. The method includes: a) providing a sample from the subject, wherein the sample includes one or more of cervicovaginal fluid, endocervical fluid, foetal membrane fluid, amniotic fluid, serum or plasma; b) measuring the elafin concentration in said sample; and c) comparing the elafin concentration of (b) to a reference standard; wherein if the elafin concentration of (b) is elevated compared to the reference standard, then it is indicated that said subject has an increased likelihood of preterm labour. Some embodiments also relate to combination methods and certain kits.

### FIELD OF THE INVENTION

The invention relates to methods for identifying subjects having an increased risk of preterm labour.

### BACKGROUND TO THE INVENTION

Preterm birth (a consequence of preterm labour) means that a baby is born before the developing organs are mature enough to allow normal postnatal survival. Premature infants are at greater risk for short and long term complications, including disabilities and impediments in growth and mental development. Significant progress has been made in the care of premature infants, but not in reducing the prevalence of preterm labour. Premature labour is the major cause of neonatal mortality in developed countries.

More specifically, preterm labour is a condition where a baby of less than 37 weeks gestational age is born. It is a syndromic condition, in that the cause for preterm labour is in many situations unknown. Many factors appear to be associated with the development of preterm labour, making the reduction of preterm labour a challenging proposition. Some of the factors associated with preterm labour include shortening of the cervix, infection of the vaginal tract, or preterm premature rupture of membranes (PPROM).

The current gold standard for detection of preterm labour is the foetal fibronectin test. This is most commonly used from weeks 22 to 35 of gestation. There is a need in the art of an alternative marker of preterm labour. There is also a need in the art for an earlier detection of preterm labour.

Elafin is a known natural antimicrobial peptide. Elafin is known to be expressed in the vaginal tract. However, nothing was known about the temporal regulation of elafin in the prior art. Similarly, no link between elafin and the on-set of preterm labour has ever been examined in the prior art.

Tromp et al (Am. J. Obstetrics and Gynaecology 2004 vol 191 pages 1331-8) disclose that expression of proteinase inhibitor 3 (PI3, a synonym for elafin—Genbank accession number D13156) is deficient in preterm premature rupture of membranes (PPROM). This paper used a mainly molecular approach to assess elafin expression in placental tissue taken at delivery from a small number of women who delivered preterm ±PPROM. There is some elafin protein localisation work in placental tissues taken at delivery (preterm and term) which is also a focus of this paper. This paper used a molecular approach to identify gene expression (mRNA) that may be altered in women plus and minus premature rupture of membranes (PPROM) in placental tissue taken after delivery. PPROM is when the fetal membranes rupture prior to a woman going into labour. These are a subgroup of women who spontaneously deliver preterm who are thought to have infection (with chorioamnionitis only being diagnosed after examination of the placenta following delivery). This paper presents a study having a cross sectional design with women in following groups (all delivered preterm) for microarray studies:

i) Women with preterm labour without histological chorioamnionitis (n=7)
ii) Preterm labour with histological chorioamnionitis (n=7)
iii) PPROM without histological chorioamnionitis (n=7)
iv) PPROM with histological chorioamnionitis (n=7)

This paper took placental tissues and extracted mRNA to determine which mRNAs were expressed differentially between the groups. It identified PI3 as an mRNA that was potentially decreased in women with PPROM. This was confirmed in a slightly larger group of women (n=10-21; including term deliveries) by:

i) quantitative qPCR (This showed a down regulation of the PI3 mRNA)
ii) immunohistochemistry (a semi quantitative technique).

These data were interpreted as immunohistochemistry demonstrating decreased proteinase inhibitor 3 protein expression in preterm premature rupture of membranes.

The authors clearly state in discussion that they hypothesise that a reduction in PI3 production will increase predisposition to PPROM.

It is to be noted that the placental tissue samples were all taken at delivery, so the inference cannot be about early pregnancy prediction, but only about diagnosis and mechanistic information about the potential cause of infection. The main inference appears to be that a reduction in uterine tissue PI3 (elafin) is associated with PPROM with chorioamnionitis, i.e. that women with an infection-linked cause of PTL have low PI3 levels in uterine tissues. This study uses a genomic (molecular/nucleic acid) approach, and does not present any protein based measurements.

King et al., (Placenta 2006) assessed elafin expression in placental and fetal membrane tissue (using semi-quantitative immunohistochemistry) taken from healthy women at term. This paper stated a working hypothesis that natural antimicrobial peptides would be altered in pregnancy associated with preterm birth (this definition encompassed pre-elampsia and spontaneous preterm birth). In the introduction to the paper evidence is quoted that reduced expression of SLPI and elafin (PI3) is associated with PPROM and then subsequently investigated:

i) localisation of human beta defensins (HBD) isoforms 1-3 and elafin protein expression in human placenta and fetal membranes (chorio-decidual) tissues;
ii) in vitro studies using placental and chorion cells in culture.

All samples in the study came from healthy women who delivered at term (n=9-10). Elafin and HBD1-3 were detected in placental tissue and localised to the syncytiotrophoblast and blood vessels. Also detected in chorion trophoblast of term fetal membranes, amnion and decidua. No obvious differences between term non-labour and term labour samples were shown. In cultured placental and chorion cells in vitro, only the inflammatory cytokine II-1 b induced elafin; TNFalpha and lipopolysaccaride did not. There is no disclosure of any inference or prediction of risk of pre-term labour.

Helmig et al (B. R. Helmig, R. Romero, J. Espinoza, T. Chaiworapongsa, E. Bujold, R. Gomez et al. Neutrophil elastase and secretory leukocyte protease inhibitor in prelabor rupture of membranes, parturition and intra-amniotic infection J Matern Fetal Neonatal Med, 12 (2002), pp. 237-246.) disclose data that amniotic fluid elafin concentrations were raised in women at delivery preterm and term in association with chorionamnionitis. There is no prediction of preterm labour. Chorionamnionitis is an inflammatory condition of the foetal membranes and is typically diagnosed or defined by examination of the placenta after birth. In other words, diagnosis is retrospective. This condition and this study have no connection to prediction, but relate only to association after parturition.

Stock et al (Stock S J, Duthie L, Tremaine T, Calder A A, Kelly R W, Riley S C. Elafin (SKALP/Trappin-2/proteinase inhibitor-3) is produced by the cervix in pregnancy and cervicovaginal levels are diminished in bacterial vaginosis. Reprod Sci. 2009) measured elafin in cervico-vaginal fluid samples from women with uncomplicated pregnancies (i.e. all delivered at term). Some of the women had bacterial vaginosis (‘BV’). BV has been linked with preterm labour but is not predictive of same. As evidenced in this study, having BV does not indicate premature delivery. In any case, in this study the women delivering at term with BV have lower elafin levels than others without BV.

Chowdhury et al., BMC Genetics 2006 discloses single nucleotide polymorphisms (molecular biomarker approach) of the peptidase inhibitor 3 (known as elafin) in women with preterm premature rupture of membranes. This document stresses the link between low PI3 and chorionamnionitis in women delivering preterm. This again advances the idea that women at most risk of PPROM have low elafin levels.

No link between elafin and preterm labour is known in the art.

The present invention seeks to overcome problem(s) associated with the art.

### SUMMARY OF THE INVENTION

Thus, the inventors have surprisingly discovered that elafin levels in the vaginal tract can be indicative of a risk of preterm labour. More importantly, the inventors have shown that elevated elafin levels in the vaginal tract during gestation can be indicative of an elevated risk of cervical shortening and/or preterm labour. Based on these surprising findings, the inventors provide a method for determining the risk of preterm labour by assessing the elafin levels in an appropriate sample such as cervico-vaginal fluid during gestation.

The present invention is distinct from Stock et al especially since (at most) Stock et al would suggest that women with BV have reduced cervico-vaginal concentrations of elafin.

The main conclusion of King et al is that antimicrobials are present in the placenta and fetal membranes. The in vitro cell data suggests they are up-regulated by one out of three pro-inflammatory cytokines applied in vitro (and even then it should be noted that the concentrations used are much higher than the inventors have ever detected in cervico-vaginal fluid). They use the observation that induction of elafin mRNA (they did not produce any data confirming that protein expression would be increased) would result in increased elafin production and that this may occur in vivo in the placenta and fetal membrane tissues in the presence of intrauterine infection.

This does not suggest in any way that in early pregnancy that elafin concentrations in a completely different region of the reproductive tract (i.e. cervico-vaginal tract/fluid) would be raised in the absence of infection (i.e. symptoms). Even less could this possibly infer that it could be used as a predictor of a short cervix or spontaneous preterm birth in high risk women. In addition, this prior art study focuses on in vitro cultured cells which have little relevance to the invention especially in preferred embodiments such as when the sample is human cervico-vaginal fluid. In addition, the non-in-vitro elements of the study were on women who delivered at term—clearly it is not possible to make inference(s) about what happens in women with preterm labour when the study is of women who delivered at term. Therefore King et al do not provide any teaching towards the present invention.

Tromp et al do not teach or infer any way of identifying women earlier in pregnancy at high risk of cervical shortening or spontaneous preterm delivery per se or indeed as a way of identify women with PPROM from those who deliver preterm. Tromp et al does not relate to cervico-vaginal fluid or early gestation events, which events could never be detected in their studies of placenta taken at delivery. In summary, Tromp et al present no evidence nor refer to any ideas relating to use of PI3/elafin protein measurements (e.g. using ELISA-based techniques) in cervico-vaginal fluid from early pregnancy gestations. The skilled person could not infer from their paper that raised levels of elafin in (e.g.) cervico-vaginal fluid would identify women who developed a short cervix or delivered spontaneously (whether+ or − PPROM). Thus the invention is surprising in the context of Tromp et al.

None of the prior art above relates to the prediction of cervical shortening or risk of spontaneous preterm birth, which is the focus of the invention. The inventors use a protein quantitative based (ELISA) approach to measure longitudinal elafin protein in a pre-term sample (such as cervico-vaginal fluid) rather than a tissue taken after delivery. The inventors teach that the sample should be from early in pregnancy and before delivery (preferably 28 weeks or earlier as described below). The inventors back up their teachings with data from a large group of women (see examples and description below). The inventors clearly show predictive benefits of sampling (e.g. CVF) for identification of high risk women with a short cervix and/or risk of spontaneous delivery. The data provided even include women with PPROM. Therefore the invention is a step change compared to prior art techniques, and this is illustrated with extensive data showing working of the invention in the application as filed.

Thus it can be clearly appreciated that the present invention is distinct from and provides demonstrable benefits over the prior art.

Thus, in one aspect, the invention provides a method of determining a risk of preterm labour in a subject, the method comprising;

a) providing a sample from said subject, wherein said sample comprises one or more of cervicovaginal fluid, endocervical fluid, foetal membrane fluid, amniotic fluid, serum or plasma;
b) measuring the elafin concentration in said sample;
c) comparing the elafin concentration of (b) to a reference standard;
wherein if the elafin concentration of (b) is elevated compared to the reference standard then it is indicated that said subject has an increased likelihood of preterm labour.

Suitably the sample comprises one or more of cervicovaginal fluid, endocervical fluid, foetal membrane fluid, amniotic fluid, serum or plasma. More suitably the sample comprises one or more of cervicovaginal fluid, endocervical fluid, foetal membrane fluid, serum or plasma. More suitably the sample comprises one or more of cervicovaginal fluid, endocervical fluid, foetal membrane fluid, or amniotic fluid. Still more suitably the sample comprises one or more of cervicovaginal fluid, endocervical fluid, or foetal membrane fluid. More suitably the sample comprises one or more of cervicovaginal fluid, endocervical fluid or foetal membrane fluid. More suitably the sample comprises one or more of cervicovaginal fluid or endocervical fluid. Most suitably the sample comprises cervicovaginal fluid (CVF).

Suitably the sample is diluted to achieve an approximate elafin concentration in the range 878 to 10,000 picograms per millilitre (pg/ml) before accurate measurement of elafin concentration. More suitably the sample is diluted to achieve an elafin concentration in the range 878 to 10,000 picograms per millilitre (pg/ml) before measurement of elafin concentration. The advantage of diluting the sample in this manner before measuring elafin concentration is that the preferred mode of determining elafin concentration works optimally when the elafin concentration is within this range.

Suitably the elafin concentration is determined by ELISA.

Suitably said sample was obtained from said subject at 24 weeks or less of gestation. This has the advantage of detection of risk early in gestation.

More suitably said sample was obtained at 12-20 weeks of gestation. This has the further advantage of being markedly earlier than prior art techniques such as fibronectin measurements.

More suitably said sample was obtained at 14-20 weeks of gestation. This has the further advantage of being the most likely window of time during which women present for examination and/or sample collection.

More suitably said sample was obtained at 14, 16, 18 or 20 weeks of gestation. Most suitably said sample was obtained at 14 weeks of gestation. It is surprising to the inventors that detection so early in pregnancy could be accomplished in this manner. Detection at (e.g.) 14 weeks is a remarkable advantage which had benefits such as enabling early intervention, and/or enabling early implementation of monitoring by other techniques such as scanning of the cervix.

In another aspect, the invention relates to a method of treating a subject at risk of preterm labour, said method comprising performing a method as described above wherein if the subject is found to have an increased likelihood of preterm labour then cervical cerclage or vaginal progesterone is administered to said subject.

Suitably the subject is at high risk of preterm labour. This has the advantage of early detection to maximise the chance of improving outcomes.

Suitably the subject is at low risk of preterm labour. This has the advantage of a low cost test which can be applied to low risk subjects, thereby identifying possible need for intervention in a wider pool of patients at a lower cost than prior art techniques.

Suitably the subject has bulging membranes.

It may be advantageous to combine the methods of the invention with one or more alternate method (s) known to be effective in detecting preterm labour. The results of a combined test have the advantage of greater certainty than the results of either single test in isolation. Thus, in another aspect, the invention relates to a method of determining the risk of preterm labour in a subject, a method comprising; a) performing a method according to any preceding claim; and b) measuring the foetal fibronectin level in the sample; comparing the level of fibronectin determined with the level of fibronectin in a reference sample; and c) if the subject has elevated elafin relative to the reference sample and has elevated fibronectin relative to the reference sample then it is indicated that said subject has an increased likelihood of preterm labour.

The invention can be applied in the monitoring of an individual patient over time. In this embodiment the ‘reference sample’ is not used, but rather the elafin concentration of the test sample (second sample) is compared to the elafin concentration of an earlier sample (first sample) from the same patient. In this way, any change in elafin concentration can be detected without need for a reference sample from a third party source. In this way, any developing elevation of elafin levels can be detected specifically for the patient being tested. Thus in one embodiment the invention provides a method of determining a risk of preterm labour in a subject, the method comprising:

a) providing a first sample from said subject, wherein said first sample comprises one or more of cervicovaginal fluid, endocervical fluid, foetal membrane fluid, amniotic fluid, serum or plasma; wherein said first sample was obtained at a first stage of gestation,
b) measuring the elafin concentration in said first sample;
c) providing a second sample from said subject, wherein said second sample comprises one or more of cervicovaginal fluid, endocervical fluid, foetal membrane fluid, amniotic fluid, serum or plasma; wherein said second sample was obtained at a second stage of gestation, said second stage of gestation being later than said first stage of gestation,
d) measuring the elafin concentration in said second sample;
e) comparing the elafin concentration of (d) to that in (b),
wherein if the elafin concentration of (d) is elevated compared to that in (b), then it is determined that that said subject has an increased likelihood of preterm labour.

In another aspect, the invention relates to a method of aiding the diagnosis of a short cervix in a subject, the method comprising:

a) providing a first sample from said subject, wherein said first sample comprises one or more of cervicovaginal fluid, endocervical fluid, foetal membrane fluid, amniotic fluid, serum or plasma; wherein said first sample was obtained at a first stage of gestation,
b) measuring the elafin concentration in said first sample;
c) providing a second sample from said subject, wherein said second sample comprises one or more of cervicovaginal fluid, endocervical fluid, foetal membrane fluid, amniotic fluid, serum or plasma; wherein said second sample was obtained at a second stage of gestation, said second stage of gestation being later than said first stage of gestation,
d) measuring the elafin concentration in said second sample;
e) comparing the elafin concentration of (d) to that in (b),
wherein if the elafin concentration of (d) is elevated compared to that in (b), then it is determined that that said subject has an increased likelihood of a short cervix.

In another aspect, the invention relates to a method of aiding the diagnosis of a short cervix in a subject, the method comprising;

a) providing a sample from said subject, wherein said sample comprises one or more of cervicovaginal fluid, endocervical fluid, foetal membrane fluid, amniotic fluid, serum or plasma;
b) measuring the elafin concentration in said sample;
c) comparing the elafin concentration of (b) to a reference standard;
wherein if the elafin concentration of (b) is elevated compared to the reference standard then it is indicated that said subject has an increased likelihood of a short cervix.

In another aspect, the invention relates to a method of determining a risk of preterm labour in a subject, the method comprising performing a method as described above wherein if the subject is determined to have an increased risk of a short cervix then the subject is scanned periodically thereafter to monitor cervical length, wherein a short cervix indicates an increased likelihood of preterm labour.

At certain elafin levels, it may not be necessary to make relative comparisons to a reference sample or to an earlier sample from the same subject. At certain elafin levels, the absolute concentration of elafin itself can be indicative of an outcome or an increased risk of a particular outcome.

Thus, in another aspect, the invention relates to a method of determining a risk of preterm labour in a subject, the method comprising;

a) providing a sample from said subject, wherein said sample comprises one or more of cervicovaginal fluid, endocervical fluid, foetal membrane fluid, amniotic fluid, serum or plasma;
b) measuring the elafin concentration in said sample;
wherein if the elafin concentration of (b) is 200 ng/ml or more then it is indicated that said subject has an increased likelihood of preterm labour.

In another aspect, the invention relates to a method of aiding the diagnosis of a short cervix in a subject, the method comprising;

a) providing a sample from said subject, wherein said sample comprises one or more of cervicovaginal fluid, endocervical fluid, foetal membrane fluid, amniotic fluid, serum or plasma;
b) measuring the elafin concentration in said sample;
wherein if the elafin concentration of (b) is 200 ng/ml or more then it is indicated that said subject has an increased likelihood of a short cervix.

Suitably the sample comprises cervicovaginal fluid.

Suitably the sample is diluted to achieve an approximate elafin concentration in the range 878 to 10,000 picograms per millilitre (pg/ml) before accurate measurement of elafin concentration. More suitably the sample is diluted to achieve an elafin concentration in the range 878 to 10,000 picograms per millilitre (pg/ml) before measurement of elafin concentration.

Suitably said sample was obtained at 14 weeks of gestation.

In another aspect, the invention relates to a diagnostic kit for assessing risk of preterm labour which comprises at least one quantifiable ligand for elafin and at least one quantifiable ligand for another marker of preterm labour such as fibronectin.

Suitably the invention provides a diagnostic kit for assessing risk of preterm labour which comprises quantifiable ligands for elafin and for fibronectin.

The ligands can be any molecule that would bind to elafin or to other markers for preterm labour. Suitable ligands include antibodies, preferably monoclonal antibodies because of their reliability and because their use as ligands for diagnostic purposes is already well established. The ligands may be suitably labelled so as to allow for the quantification of the ligand, and therefore the concentration of elafin and other makers of preterm labour. One example of a labelled ligand is a fluorescently tagged monoclonal antibody. Alternatively the kit may comprise additional reagents for detection of bound ligands. The kit may further comprise means for taking a sample from a subject; for example, swabs, syringes, or the like.

### DETAILED DESCRIPTION OF THE INVENTION

In a strict sense, preterm labour leads to preterm birth. However, in practice the terms are often used interchangeably. Sometimes preterm labour or preterm birth are referred to as ‘spontaneous’, thus preterm labour may also be known as spontaneous preterm labour and preterm birth may be known as spontaneous preterm birth (SPTB).

Suitably the sample is an in vitro sample. Suitably the sample has been previously collected from the subject. Suitably the sample is provided as an in vitro sample. Suitably the methods of the invention do not include collection of the sample from the subject. Suitably the methods of the invention are conducted in a laboratory. Suitably the methods of the invention do not involve the presence of the subject. Suitably the step of sample collection is specifically excluded from the methods of the invention.

Suitably measuring the elafin concentration is carried out by measuring elafin protein levels. Suitably elafin is assayed by detection/quantification of elafin polypeptide. Suitably the assay does not involve assessment of elafin nucleic acid (s).

Suitably the methods of the invention are in vitro methods. Suitably the methods of the invention are methods of aiding a diagnosis. Suitably the methods of the invention provide information which can be used by a physician in arriving at a diagnosis.

Suitably the methods may be information based methods for aiding the diagnosis of a subject. Suitably the methods do not involve the presence of the subject. Suitably the methods are conducted only on a sample previously obtained from the subject. In these embodiments ‘determining a risk’ may instead be ‘aiding a diagnosis’ or ‘aiding a diagnosis of increased risk’.

### DEFINITIONS

The term ‘comprises’ (comprise, comprising) should be understood to have its normal meaning in the art, i.e. that the stated feature or group of features is included, but that the term does not exclude any other stated feature or group of features from also being present.

A woman is at high risk of preterm labour if that woman has had one or more previous second trimester losses. A woman is at high risk of preterm labour if that woman has had a previous preterm labour such as a previous spontaneous preterm labour. A woman is at high risk if that woman has experienced both one or more previous second trimester losses and a previous preterm labour.

A woman is at low risk of preterm labour if she has previously had one or more births at 37 weeks of gestation or later.

A woman is at high risk of preterm labour if that woman has a short cervix before 24 weeks of gestation.

A short cervix is a cervix of less than 25 mm.

A short cervix may be a cervix of 15 mm or less.

A short cervix precedes preterm labour. However, not all short cervixes result in preterm labour. A short cervix can occur several weeks before preterm labour may begin.

It is advantageous to detect or predict a short cervix because short cervix identifies subjects who are at risk, and/or should have regular monitoring. Short cervix also indicates a high risk of preterm labour.

If short cervix is detected or predicted according to the present invention, advantageously that subject is referred for monitoring thereafter. Monitoring may be by cervical scanning.

The subject is suitably a mammal, more suitably a primate. Most suitably the subject is a human.

Preterm labour (sometimes called premature labour) is defined as labour before 37 weeks of gestation. When the subject is a human, the term “preterm labour” preferably means labour at less than 37 weeks' completed gestation. Preferably, it means at between 16 and 37 weeks' completed gestation, more preferably between 20 and 37 weeks' completed gestation especially between 24 and 37 weeks' completed gestation. Gestational age may be assessed using the last menstruation date and ultrasound examination.

Suitably preterm labour is at less than 37 weeks' gestation.

Suitably preterm labour is at less than 34 weeks' gestation.

Suitably preterm labour is at less than 32 weeks' gestation.

The tests of the invention are still useful in the 25^{th} or subsequent weeks of gestation. For example, the tests can be useful at these later times for clinical management. However, the key advantages of the invention are in the predictive value and the early stage risk assessment which has not been possible in the prior art. The extra benefits of the invention are therefore reaped by carrying out the tests on samples from 24^{th} or earlier weeks of gestation. One reason is that from a clinical point of view, surveillance clinics tend to discharge their patients from 24 weeks onwards, since at this point in gestation the foetus is typically viable. Therefore the invention is particularly useful when applied to samples collected from the 24^{th} and earlier weeks of gestation as described. In addition, the test is especially useful in the 21^{st} and earlier weeks because the prior art foetal fibronectin test is typically only effective from 22 weeks onwards. In any case, the invention still presents benefits over the foetal fibronectin test because that has a low sensitivity, identifying only about 60% of patients with problems, whereas the tests of the invention are advantageously more effective.

### Reference Standard

The sample may be compared with a reference standard or reference sample from another subject. If the concentration of elafin is higher than that of the reference standard, the level may be considered to be increased (elevated).

The sample may be compared with a standard level or range of levels appropriate for the sample type and the gestation at which the sample is collected. Reference ranges may be established by testing a suitable number of comparable samples from a number of subjects over a range of gestational ages. Such reference ranges may allow the determination of the distribution of concentration of elafin at a given gestation.

Suitably the reference standard is age matched to the subject.

Suitably the reference standard is ethnicity matched to the subject.

Suitably the reference standard is matched for time of gestation to the subject.

Comparable samples are the same type of sample as each other. For example, comparable samples are taken from subjects of the same species. Equally, blood samples should be compared with other blood samples, CVF samples with other CVF samples etc.

The reference standard need not necessarily involve the parallel testing of another sample alongside the testing of the sample from the subject of interest. The reference standard may in fact be a numerical value determined on a past occasion. Therefore the comparison with the reference standard may be a purely numerical exercise of comparing the two determined values.

When compared with a sample from another subject or with a standard level, the increase in the concentration of elafin is suitably a significant increase. The elafin concentration is preferably at least twice, more preferably at least four times the expected concentration. Alternatively, in the instance that the presence of elafin in the sample is not expected, the simple detection of elafin could be considered to be an increase in the terms of the invention.

Alternatively, the sample may be compared with a projected or likely concentration of elafin, established by testing the same subject at an earlier date and predicting the concentration of elafin that might be reached by the relevant stage of gestation. The sample may also simply be compared with earlier samples obtained from the subject to identify a change, especially an increase in concentration of elafin.

When testing samples from the same subject, the samples may be obtained repeatedly, such as daily, every two or three days, weekly, fortnightly or at longer intervals.

Most suitably the reference standard is the value of 200 ng/ml of cervico-vaginal fluid (CVF) from a human.

### Elafin

Elafin is an epithelial proteinase inhibitor also known as Skin-derived Anti leukoproteinase (SKALP) and Elastase-Specific Inhibitor (ESI) and Trappin 2 as elafin belongs to the Trappin gene family. The Trappin family is defined by a N-terminal transglutaminase substrate domain and a C-terminal four disulphide core. Trappins play a role in the regulation of inflammation and in protection against tissue damage in stratified epithelia. Elafin is an inhibitor of leukocyte elastase and proteinase-3 and in addition it is a substrate for transglutaminases. The protein is constitutively expressed in various epithelia including those of hair follicles, esophagus, vagina and oral cavity. Elafin production has been demonstrated to be induced by vitamin D in vitro. The full-length protein is translated as a 12.3 kDa protein of 117 amino acids termed pre-elafin or Trappin-2. Cleavage of the signal peptide yields a mature protein with a molecular mass of 9.9 kDa. The 9.9 kDa secreted protein is the major form found in culture medium. In skin extracts a 6 kDa form comprising the 57 most C-terminal amino acids is present, which is the form of elafin originally described by Wiedow et al. In serum both the 9.9 and the 6 kDa form appear to be present. In urine only the short 6 kDa form is found.

The reference sequence for elafin is provided below.

When particular amino acid residues are referred to using numeric addresses, the numbering is taken using the elafin amino acid sequence as the reference sequence. This is to be used as is well understood in the art to locate the residue of interest. This is not always a strict counting exercise—attention must be paid to the context. For example, if the protein of interest is of a slightly different length, then location of the correct residue in the sequence corresponding to (for example) the 21^{st} residue may require the sequences to be aligned and the equivalent or corresponding residue picked, rather than simply taking the 21^{st} residue of the sequence of interest. This is well within the ambit of the skilled reader.

Elafin is a known protein. The Generic Protein details are:

Elafin [Homo sapiens]
21 amino acids protein

### Genbank Accession:AAB34627

The specific version at the date of filing is represented by Genbank Accession:AAB34627.1; GI:999146.

### Full Length Elafin Accession Numbers:

elafin preproprotein [Homo sapiens]
117 aa protein

### NP_{—}002629.1 GI:4505787

elafin precursor [Homo sapiens]
117 aa protein

### AAA36483.1 GI:190338

PI3 [Homo sapiens]
117 aa protein

### CAG47030.1 GI:49457462

It should be noted that the invention is concerned with the detection of elafin protein i.e. expressed elafin polypeptide. Nevertheless in case it is any help to the skilled reader, we also provide the nucleic acid sequences encoding the elafin protein:

### PI3

Official Symbol: PI3 and Name: peptidase inhibitor 3, skin-derived [Homo sapiens]

### Other Aliases:

ESI, SKALP, WAP3, WFDC14, cementoin
Other Designations: PI-3; WAP four-disulfide core domain 14; WAP four-disulfide core domain protein 14; elafin; elastase-specific inhibitor; pre-elafin; protease inhibitor 3, skin-derived (SKALP); protease inhibitor WAP3; skin-derived antileukoproteinase; trappin-2 Chromosome: 20; location: 20q13.12 Annotation: Chromosome 20, NC_{—}000020.10 (43803540 . . . 43805185)

### MIM:182257

### ID:5266

### SLPI

Official Symbol: SLPI and Name: secretory leukocyte peptidase inhibitor [Homo sapiens]

### Other Aliases: ALK1, ALP, BLPI, HUSI, HUSI-I, MPI, WAP4, WFDC4

Other Designations: HUSI-1; WAP four-disulfide core domain protein 4; antileukoproteinase; mucus proteinase inhibitor; protease inhibitor WAP4; secretory leukocyte protease inhibitor (antileukoproteinase); seminal proteinase inhibitor Chromosome: 20; Location: 20q12 Annotation: Chromosome 20, NC_{—}000020.10 (43880879 . . . 43883206, complement)

### MIM: 107285 ID:6590

The elafin concentrations measured in the methods according to the present invention can be measured with any assay technique known in the art. It is advantageous if the elafin concentrations which are to be compared with each other are measured with the same assay technique.

As the elevated elafin concentration is defined in the present invention by comparison, it is preferable that the concentration values of elafin are measured in the most standardised fashion possible. It is therefore preferable to use the same technique to take different measurements. There are many techniques known in the art to measure the concentration of a protein such as elafin in a sample. An example of a suitable assay technique to be used is ELISA (enzyme linked immunosorbent assay).

The collection of the measurements taken can be improved by diluting the sample as necessary before taking the measurement. The sample should be diluted with buffer suitable to the assay technique. When using ELISA as the assay technique, the most accurate readings are taken when the concentration of elafin is in the range of 878-10,000 pg/ml. It is therefore suitable to dilute the sample (for example by 1:10 to 1:100) with assay buffer before carrying out the ELISA assay. Any such dilutions must of course be taken into account in calculating the concentration of elafin in the sample.

The preferred mode of detecting elafin is to use the commercially available HK318 human elafin ELISA kit from Hycult Biotech, Frontstraat 2a, 5405 PB Uden, Netherlands. Details may be easily found here http://www.hycultbiotech.com/neutrophil-proteins/elisa-kit-for-human-elafin-p10922.html. This kit is suitably used according to the manufacturer's instructions unless otherwise indicated herein. The manufacturer's instructions in their published document ‘Manual HK318 (Edition 03-10) are specifically incorporated herein by reference for the detail of the conduct of the ELISA assay to which it relates. In more detail, section 7 (sample preparation), section 8 (reagent preparation) and section 9 (ELISA protocol) of this document are specifically incorporated herein by reference for the preferred conduct of the ELISA test.

The HK318 ELISA test detects 2 forms of elafin. There is a pre-elafin protein TRAPPIN-2 and the cleaved protein elafin (also known as Trappin2/elafin).

The manufacturer explains as follows: The full-length protein is translated as a 12.3 kDa protein of 117 amino acids termed pre-elafin or Trappin-2. Cleavage of the signal peptide yields a mature protein with a molecular mass of 9.9 kDa. The 9.9 kDa secreted protein is the major form found in culture medium.

If it is desired to determine which of these proteins predominates in the sample such as cervical fluid, this can be ascertained using Western blot analysis.

Without wishing to be bound by theory, it seems that the signal is likely to be ‘mixed’ i.e.

detecting both pre-elafin and elafin. For the purposes of the invention the precise elafin species which is detected is not considered relevant. For the avoidance of doubt, ‘elafin’ should be understood to mean the species detected by the preferred HK318 elafin ELISA test.

When an antibody is used to detect elafin, then suitably any antibody capable of specifically recognising elafin may be employed. Suitably the antibody is as described in the HK318 kit above.

### Sample

The sample collection is conducted according to standard methods well known in the art according to the sample type (composition).

Especially suitable methods include the following:

### Vaginal Swab Sample Collection and Preparation

### Collection of Vaginal Swabs—Cervicovaginal Fluid

The subject was asked to lie on the couch with a sheet placed over her abdomen. The subject was asked to place the soles of her feet together and draw her knees up towards her abdomen. The labia were gently parted and the vaginal speculum was gently inserted into the vagina. Swabs for clinical use were taken first if indicated, followed by the high vaginal swab and the endocervical swab (if required). When swabs were taken for research purposes, they were rotated in the posterior vaginal fornix (HVS) or the endocervix (ECS) for 10 seconds to achieve saturation. Once this was complete, the speculum was gently removed and excess lubricant was wiped away.

Two pre-labelled pre-prepared vials of 750 μl of standard phosphate-buffered saline solution containing protease inhibitor [1 protease inhibitor cocktail tablet (Complete, Roche Diagnostics GmbH, Germany) dissolved in 50 ml standard phosphate-buffered saline solution (Sigma-Aldrich Company, Ayrshire, UK] were gently warmed to room temperature for a few seconds. The swab tips were placed in the vials and then on ice for immediate transport to the laboratory or placed in the centrifuge and prepared prior to being stored at −80° C. Following the procedure, all used equipment was disposed of appropriately.

The procedure for collection of endocervical fluid is the same as above, with the modification that the swab is inserted into the cervical canal.

Reparation of Swab Samples from High Vagina (Cervicovaginal Fluid) or Endocervix (Endocervical Fluid)

The swab tip was removed and placed into a clean empty tube. The tube containing the vaginal or cervical mucus in phosphate-buffered saline solution with protease inhibitor was vortexed for 30 s and placed on ice. The tube containing the Dacron swab tip was centrifuged at 2,600 g (4,000 rpm) for 10 minutes at 4° C. on a standard centrifuge (Centrifuge 5702R, Eppendorf, UK) to collect any extra mucus that was adherent to the tip. The extra sample was collected and pipetted into the eppendorf tube containing the cervical mucus sample. The resultant fluid was vortexed for 30 s and then 110 μl was aliquotted into 2 ml plastic microfuge tubes (high vaginal sample and endocervical samples). The samples were frozen at −80° C. until analysis.

### Foetal Outcomes

In one aspect the invention relates to a method of predicting foetal outcomes for a subject, the method comprising;

a) providing a sample from said subject, wherein said sample comprises one or more of cervicovaginal fluid, endocervical fluid, foetal membrane fluid, amniotic fluid, serum or plasma;
b) measuring the elafin concentration in said sample;
c) comparing the elafin concentration of (b) to a reference standard;
wherein if the elafin concentration of (b) is elevated compared to the reference standard then it is indicated that said subject has an increased likelihood of a poor foetal outcome.

A poor foetal outcome may be a foetal infection.

A poor foetal outcome may be infection associated morbidity.

A poor foetal outcome may be an infected baby at birth.

In other words, the methods of the invention may be used to predict elevated risk of an infected foetus, or an infected baby at birth.

If an elevated risk of poor foetal outcome is detected, then it may influence the treatment or lack of treatment administered to the subject.

For example the invention may encompass a method of aiding the decision how to treat a subject comprising performing the method as described above wherein if the subject has an increased likelihood of a poor foetal outcome then it is indicated that interventions to extend pregnancy (i.e. to prevent preterm birth) may be avoided. For example if an increased likelihood of foetal infection is detected, then it may be indicated to avoid or decide against treatment by insertion of a stitch such as a cervical stitch (e.g. cervical cerclage). The reasoning is that if there is a greater chance of intrauterine infection, then extending the pregnancy or preventing preterm birth may lead to a poorer outcome than allowing the baby to be born preterm.

### Combinations

The methods described above are advantageously carried out together with measurement of other markers of preterm labour. The quantification of other markers can be carried out on the same sample or on a separate sample obtained at the same time. If the sample is the same, the assay technique can be different to the one used for elafin. If necessary, the sample may be divided. If the assay technique is the same, the quantification of the markers can take place together e.g. simultaneously.

Suitable markers which indicate increased likelihood of preterm labour which might be measured with elafin in such a combination method include one or more of: elevated levels of fibronectin, elevated levels of endocannabinoid such as anandamide, elevated levels of MCP-1, elevated levels of GM-CSF, or decreased levels of progesterone. The preferred marker is fibronectin, as it is already well established in clinical practice.

In some aspects of the invention, it may be advantageous to combine the invention with a further test such as the foetal fibronectin test. Thus, in another aspect the invention relates to performing a method as described above, and further conducting a foetal fibronectin test.

### Quantification of Risk

In some embodiments the values obtained for the elafin concentration measurements may be used to quantify the risk of preterm labour (and/or short cervix).

Data presented herein may be used to infer the particular risks from the particular values obtained.

### Further Applications and Advantages

In one application the invention allows significant cost and labour savings by focussing resources on women in need or at risk of preterm labour. Currently cervical scanning may be undertaken for large cohorts of women. Suitably according to the present invention women are tested using the elafin based methods of the invention. Then those women found to be at increased risk/increased likelihood of preterm birth and/or short cervix can be entered into a regular screening such as by cervical monitoring.

### BRIEF DESCRIPTION OF THE FIGURES

FIG. 1 shows a diagram of Enrollment, randomization and follow-up of participants to original CLIC study.

FIG. 2 shows typical standard curve (taken from Hycult Biotech product information)

FIG. 3 shows Cervico-vaginal elafin concentrations across gestation in women without a short cervix (controls) and women destined to get a short cervix between 14 and 28 weeks' (cases). All data are analysed including when women are on treatment (n=324 uncensored, 85 right censored, 28 left censored observations; 437 in total, on 74 women).

FIG. 4 shows Cervico-vaginal fluid concentrations predict cervical shortening at 14 weeks'. Panel A: ROC curve (area under the curve is 1.00; CI: 1.00 to 1.00; n=11, P=8.2e-03). Panel B: Dot blot (0=women who do not exhibit a short cervix by 24 weeks' and 1=women who develop a short cervix before 24 weeks')

FIG. 5 shows Cervico-vaginal fluid concentrations predict cervical shortening at 16 weeks'. Panel A: ROC curve (area under the curve is 0.85 CI: 0.71 to 0.98; n=42, P=1.8e-04). Panel B: Dot blot (0=women who do not exhibit a short cervix by 24 weeks' and 1=women who develop a short cervix before 24 weeks')

FIG. 6 shows Cervico-vaginal fluid concentrations predict cervical shortening at 18 weeks'. Panel A: ROC curve (area under the curve is 0.82 CI: 0.67 to 0.98; n=40, P=1.4e-04). Panel B: Dot blot (0=women who do not exhibit a short cervix by 24 weeks' and 1=women who develop a short cervix before 24 weeks')

FIG. 7 shows Cervico-vaginal fluid concentrations predict cervical shortening at 20 weeks'. Panel A: ROC curve (area under the curve is 0.82 CI: 0.63 to 1.00; n=39, P=5.9e-03). Panel B: Dot blot (0=women who do not exhibit a short cervix by 24 weeks' and 1=women who develop a short cervix before 24 weeks')

The invention is now described by way of example. These examples are intended to be illustrative, and are not intended to limit the appended claims.

### EXAMPLES

### Example 1

### Methods

Elafin (and serum 25-Hydroxy Vitamin D) were measured in a subset of cervico-vaginal and blood samples were analysed obtained as part of a prospective longitudinal observational study (CLIC, STH Ethics Approval No: 06/Q0704/66) which assessed 112 women with at least one previous preterm delivery between 16 and 34 weeks' gestation. Exclusion criteria included multiple pregnancies, previous iatrogenic preterm births and inability to give informed consent.

Recruits were initially assessed every 2 weeks by transvaginal cervical length assessment as well as cervico-vaginal fluid and blood sampling between 14 and 28 weeks' gestation. All women provided a cervico-vaginal fluid sample prior to transvaginal cervical length assessment at every visit. If the cervical length shortened to less than 25 mm before 24 weeks' gestation, women received a routine intervention of either cervical cerclage or vaginal progesterone. Samples and scans were repeated weekly thereafter. Natural progesterone Cyclogest® 400 mg once daily (Actavis UK Ltd, Devon, UK) was used based on current clinical practice in light of insufficient contemporary evidence of optimal route and dose of progesterone administration.

Women were randomly assigned to treatments for the purpose of ensuring equal numbers in each intervention group. Computer generated open-label randomization in blocks of 8, prepared by the unit data manager (otherwise uninvolved in the study) was undertaken. The participants were enrolled and randomized by the study investigator, who was blinded to the randomization sequence. High-risk women were defined as controls if they did not have a short cervix before 24 weeks' gestation (n=64) and as cases if their cervix shortened to <25 mm before 24 weeks' gestation (n=37). Routine screening for bacterial vaginosis was not included in the study protocol, but if women presented with suggestive symptoms a high vaginal swab was taken; those testing positive received clindamycin (6/18 controls and 10/31 cases).

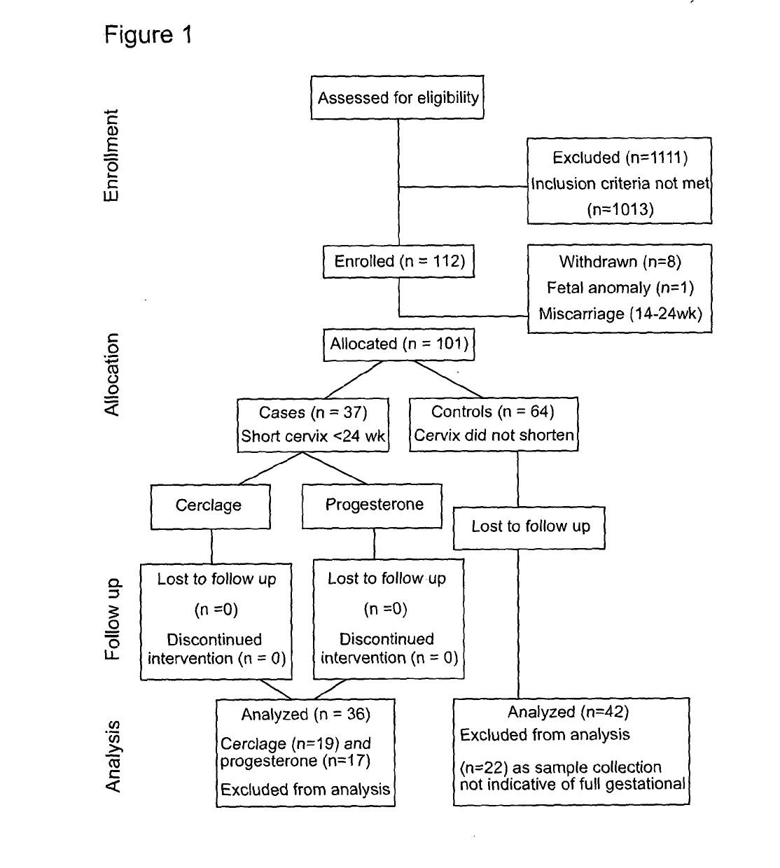

See FIG. 1 for a diagram of the study protocol.

Elafin concentrations were measured in cervico-vaginal fluid samples (437 samples from 74 women, approx 5.91 observations per women; n=38 controls and n=36 cases) using a commercially available Human elafin ELISA kit (Hycult Biotech, The Netherlands, HK318). The ELISA kit detects the 9.9 kDa cleaved form of the 12.3 kDA protein known as pre-elafin or Trappin-2. It does not detect the 6 kDa from present in the skin and urine. Cervico-vaginal fluid samples were diluted (between 1:10 to 1:100) with assay buffer to ensure that the majority of the samples were detected by the assay (measurable concentration range of 878-10,000 pg/ml).

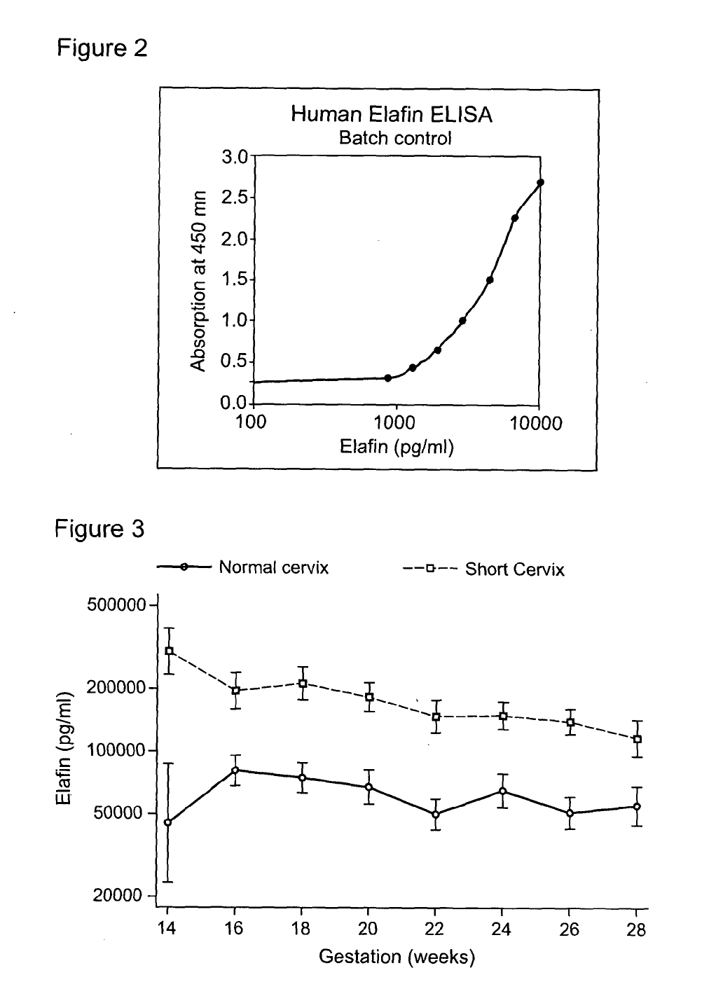

Principle of the assay (see Hycult Biotech product information): The human elafin ELISA is a ready-to-use solid-phase enzyme-linked immunosorbent assay based on the sandwich principle with a working time of 3½ hours. The format of 2 plates with twelve disposable 8-well strips allowed free choice of batch size for the assay. Samples and standards are captured by a solid bound specific antibody. Biotinylated tracer antibody binds to captured human elafin. Streptavidin-peroxidase conjugate binds to the biotinylated tracer antibody. Streptavidin-peroxidase conjugates react with the substrate, tetramethylbenzidine (TMB). The enzyme reaction is stopped by the addition of oxalic acid. The absorbance at 450 nm is measured with a spectrophotometer. A standard curve is obtained by plotting the absorbance (linear) versus the corresponding concentrations of the human elafin standards (log). The human elafin concentration of samples, which are run concurrently with the standards, are determined from the standard curve generated. See FIG. 2.

### Statistic al Analysis

The sample size was not pre-determined due to inadequate published data informing the gestational profile of cervico-vaginal fluid elafin concentrations, or of any relationship with repeated measures of cervical length. This study and analysis was therefore exploratory in nature. The predefined endpoints were the cervico-vaginal elafin concentration prior to cervical shortening, and before and after treatment. The relation between elafin concentrations and SPTB was also examined. Elafin concentrations were expressed as pg/ml. Data were analyzed using Stata (version 10.1, Stata Corp, College Station, Tex.). Distributions of data were first established by examination of distributional plots for raw and transformed values. Log transformations were used to achieve approximate normality as needed. Estimates and tests of differences, both between and within subjects, used linear regression adjusting for repeated measures and non-normality through the use of generalized estimating equations. Where sample concentrations were below the limit of detection for the assay, an interval regression method was used, with the missing values taken as being at an unknown point on the interval between zero and the smallest positive concentration observed.

Of 467 individual samples from 77 women, (3 women with missing gestation at delivery), dilutions were only conducted in the range 10× to 50×. For 31 samples, concentrations of Elafin after dilution were below the detectable limit of 878 pg/ml; of these 20 had approximate values extrapolated outside the standard curve as defined by the manufacturer. Likewise, concentrations in 96 samples were >10,000 pg/ml; 79 having extrapolated values.

Three women (19 samples) with unknown gestation at delivery were excluded. In order to simplify the analysis, second samples within a 2-week period were also excluded. This reduced the dataset to 379 samples from 74 women.

### Example 2

### Categorization (Banding) of Elafin Concentrations

Values are categorized as <50, 50-100, 100-200, or 200 ng/ml+.

We give below performance of these categories for detecting a short cervix in high risk women in 2-weekly intervals of gestation.

We also have compared the simple categories with the exact values for elafin using ROC (receiver operated characteristic curve), areas for prediction of short cervix. The example at 14 weeks is given below.

Further details are in example 3 below.

The best single cut-off is 200 ng/ml, but using exact values is best.

Results for gestation of 14 (2-week period, from 13 to 14+6).

| Key | | | | | |
|---|---|---|---|---|---|
| frequency | | | | | |
| row percentage | | | | | |
| | short_cx | | | | |
| elafin_Catagory | No | Yes | Total | | |
| <50 ng/ml | 1 | 1 | 2 | | |
| | 50.00 | 50.00 | 100.00 | | |
| 50-100 | 2 | 0 | 2 | | |
| ng/ml | 100.00 | 0.00 | 100.00 | | |
| 100-200 | 1 | 0 | 1 | | |
| ng/ml | 100.00 | 0.00 | 100.00 | | |
| 200 | 0 | 8 | 8 | | |
| ng/ml+ | 100.00 | 100.00 | 100.00 | | |
| Total | 4 | 9 | 13 | | |
| | 30.77 | 69.23 | 100.00 | | |
| | | | | -Asymptotic | |
| | | | | Normal- | |
| | | ROC | Std. | [95% Conf. | |
| | Obs | Area | Err. | Interval] | |
| elafin_val | 12 | 1.0000 | 0.0000 | 1.00000 | 1.00000 |
| elafin_cat | 12 | 1.0000 | 0.0000 | 1.00000 | 1.00000 |
| Ho: area (elafin_val) = | | | | | |
| area (elafin_cat) | | | | | |
| chi 2 (0) = 0.00 | | | | | |
| Prob > chi 2 = . | | | | | |

Predictive values of elafin test (4 categories) for prematurity (34 & 37 weeks) Results for gestation of 14 (2-week period, from 13 to 14+6):

Key
frequency
row percentage

| | del_24 | | | | |
|---|---|---|---|---|---|
| elafin_cat | NO | YES | Total | | |
| <50 ng/ml | 1 | 1 | 2 | | |
| | 50.00 | 50.00 | 100.00 | | |
| 50-100 | 2 | 0 | 2 | | |
| ng/ml | 100.00 | 0.00 | 100.00 | | |
| 100-200 | 1 | 0 | 1 | | |
| ng/ml | 100.00 | 0.00 | 100.00 | | |
| 200 | 4 | 4 | 8 | | |
| ng/ml+ | 50.00 | 50.00 | 100.00 | | |
| Total | 8 | 5 | 13 | | |
| | 61.54 | 38.46 | 100.00 | | |
| | | | | -Asymptotic | |
| | | | | Normal- | |
| | | ROC | Std. | [95% Conf. | |
| | Obs | Area | Err. | Interval] | |
| elafin_val | 12 | 1.0000 | 0.0000 | 1.00000 | 1.00000 |
| elafin_cat | 12 | 0.7500 | 0.0945 | 0.56480 | 0.93520 |
| Ho: area (elafin_val) = | | | | | |
| area (elafin_cat) | | | | | |
| chi 2 (1) = 7.00 | | | | | |
| Prob > chi 2 = 0.0082 | | | | | |

### Example 3

### Use of Elafin as a Predictor of Short Cervix (<15 mm) or Prematurity (Before 34 and 37 Weeks)

We present data for elafin predicting preterm delivery at <34 weeks (and 37 weeks).

Despite dilution, not all elafin values are measured exactly and within the intended range of the assay

These elafin values are treated in two ways, likely to resemble what might happen in practice:

a) The best approximation (even if outside the intended range) is used.
b) Values are categorized as <50, 50-100, 100-200, or 200 ng/ml+.

Where there is only a range (<878, n=8 or >10000 pg/ml, n=8) only method b) is useable; and these observations will be excluded from comparisons between a) and b).

Where 2 values are recorded in the same 2-week period, only the first is kept.

(50 observations deleted)

Predictive values of elafin test (4 categories) for short cervix (NOTE: values out of range are estimates by extrapolation, where possible)

Results for gestation of 14 (2-week period, from 13 to 14+6).

Key
frequency
row percentage

| | short_cx | | | | |
|---|---|---|---|---|---|
| elafin_cat | 0 | 1 | Total | | |
| <50 ng/ml | 1 | 1 | 2 | | |
| | 50.00 | 50.00 | 100.00 | | |
| 50-100 | 2 | 0 | 2 | | |
| ng/ml | 100.00 | 0.00 | 100.00 | | |
| 100-200 | 1 | 0 | 1 | | |
| ng/ml | 100.00 | 0.00 | 100.00 | | |
| 200 | 0 | 8 | 8 | | |
| ng/ml+ | 0.00 | 100.00 | 100.00 | | |
| Total | 4 | 9 | 13 | | |
| | 30.77 | 69.23 | 100.00 | | |
| | | | | -Asymptotic | |
| | | | | Normal- | |
| | | ROC | Std. | [95% Conf. | |
| | Obs | Area | Err. | Interval] | |
| elafin_val | 12 | 1.0000 | 0.0000 | 1.0000 | 1.0000 |
| elafin_cat | 12 | 1.0000 | 0.0000 | 1.0000 | 1.0000 |
| Ho: area (elafin_val) = | | | | | |
| area (elafin_cat) | | | | | |
| chi 2 (0) = 0.00 | | | | | |
| Prob > chi 2 = | | | | | |

Results for gestation of 16 (2-week period, from 15 to 16+6).

Key
frequency
row percentage

| | short_cx | | | | |
|---|---|---|---|---|---|
| elafin_cat | 0 | 1 | Total | | |
| <50 ng/ml | 8 | 2 | 10 | | |
| | 80.00 | 20.00 | 100.00 | | |
| 50-100 | 6 | 1 | 7 | | |
| ng/ml | 85.71 | 14.29 | 100.00 | | |
| 100-200 | 4 | 9 | 13 | | |
| ng/ml | 30.77 | 69.23 | 100.00 | | |
| 200 | 4 | 12 | 16 | | |
| ng/ml+ | 25.00 | 75.00 | 100.00 | | |
| Total | 22 | 24 | 46 | | |
| | 47.83 | 52.17 | 100.00 | | |
| | | | | -Asymptotic | |
| | | | | Normal- | |
| | | ROC | Std. | [95% Conf. | |
| | Obs | Area | Err. | Interval] | |
| elafin_val | 45 | 0.7688 | 0.0742 | 0.62336 | 0.91419 |
| elafin_cat | 45 | 0.7569 | 0.0725 | 0.61474 | 0.89909 |
| Ho: area (elafin_val) = | | | | | |
| area (elafin_cat) | | | | | |
| chi 2 (1) = 0.27 | | | | | |
| Prob > chi 2 = 0.6017 | | | | | |

Results for gestation of 18 (2-week period, from 17 to 18+6).

Key
frequency
row percentage

| | short_cx | | | | |
|---|---|---|---|---|---|
| elafin_cat | 0 | 1 | Total | | |
| <50 ng/ml | 11 | 2 | 13 | | |
| | 84.62 | 15.38 | 100.00 | | |
| 50-100 | 8 | 4 | 12 | | |
| ng/ml | 66.67 | 33.33 | 100.00 | | |
| 100-200 | 6 | 8 | 14 | | |
| ng/ml | 42.86 | 57.14 | 100.00 | | |
| 200 | 3 | 11 | 14 | | |
| ng/ml+ | 21.43 | 78.57 | 100.00 | | |
| Total | 28 | 25 | 53 | | |
| | 52.83 | 47.17 | 100.00 | | |
| | | | | -Asymptotic | |
| | | | | Normal- | |
| | | ROC | Std. | [95% Conf. | |
| | Obs | Area | Err. | Interval] | |
| elafin_val | 51 | 0.7919 | 0.0625 | 0.66952 | 0.91433 |
| elafin_cat | 51 | 0.7865 | 0.0619 | 0.66512 | 0.90787 |
| Ho: area (elafin_val) = | | | | | |
| area (elafin_cat) | | | | | |
| chi 2 (1) = 0.06 | | | | | |
| Prob > chi 2 = 0.8063 | | | | | |

Results for gestation of 20 (2-week period, from 19 to 20+6).

Key
frequency
row percentage

| | short_cx | | | | |
|---|---|---|---|---|---|
| elafin_cat | 0 | 1 | Total | | |
| <50 ng/ml | 12 | 1 | 13 | | |
| | 92.31 | 7.69 | 100.00 | | |
| 50-100 | 5 | 3 | 8 | | |
| ng/ml | 62.50 | 37.50 | 100.00 | | |
| 100-200 | 7 | 7 | 14 | | |
| ng/ml | 50.00 | 50.00 | 100.00 | | |
| 200 | 6 | 13 | 19 | | |
| ng/ml+ | 31.58 | 68.42 | 100.00 | | |
| Total | 30 | 24 | 54 | | |
| | 55.56 | 44.44 | 100.00 | | |
| | | | | -Asymptotic | |
| | | | | Normal- | |
| | | ROC | Std. | [95% Conf. | |
| | Obs | Area | Err. | Interval] | |
| elafin_val | 52 | 0.7736 | 0.0638 | 0.64849 | 0.89874 |
| elafin_cat | 52 | 0.7661 | 0.0633 | 0.64200 | 0.89024 |
| Ho: area (elafin_val) = | | | | | |
| area (elafin_cat) | | | | | |
| chi 2 (1) = 0.14 | | | | | |
| Prob > chi 2 = 0.7076 | | | | | |

Predictive values of elafin test (4 categories) for prematurely (34 & 37 weeks)

Results for gestation of 14 (2-week period, from 13 to 14+6).

Key
frequency
row percentage

| | del_34 | | | | |
|---|---|---|---|---|---|
| elafin_cat | 0 | 1 | Total | | |
| <50 ng/ml | 1 | 1 | 2 | | |
| | 50.00 | 50.00 | 100.00 | | |
| 50-100 | 2 | 0 | 2 | | |
| ng/ml | 100.00 | 0.00 | 100.00 | | |
| 100-200 | 1 | 0 | 1 | | |
| ng/ml | 100.00 | 0.00 | 100.00 | | |
| 200 | 4 | 4 | 8 | | |
| ng/ml+ | 50.00 | 50.00 | 100.00 | | |
| Total | 8 | 5 | 13 | | |
| | 61.54 | 38.46 | 100.00 | | |
| | | | | -Asymptotic | |
| | | | | Normal- | |
| | | ROC | Std. | [95% Conf. | |
| | Obs | Area | Err. | Interval] | |
| elafin_val | 12 | 1.0000 | 0.0000 | 1.00000 | 1.00000 |
| elafin_cat | 12 | 0.7500 | 0.0945 | 0.56480 | 0.93520 |
| Ho: area (elafin_val) = | | | | | |
| area (elafin_cat) | | | | | |
| chi 2 (1) = 7.00 | | | | | |
| Prob > chi 2 = 0.0082 | | | | | |

Key
frequency
row percentage

| | del_37 | | | | |
|---|---|---|---|---|---|
| elafin_cat | 0 | 1 | Total | | |
| <50 ng/ml | 1 | 1 | 2 | | |
| | 50.00 | 50.00 | 100.00 | | |
| 50-100 | 2 | 0 | 2 | | |
| ng/ml | 100.00 | 0.00 | 100.00 | | |
| 100-200 | 1 | 0 | 1 | | |
| ng/ml | 100.00 | 0.00 | 100.00 | | |
| 200 | 3 | 5 | 8 | | |
| ng/ml+ | 37.50 | 62.50 | 100.00 | | |
| Total | 7 | 6 | 13 | | |
| | 53.85 | 46.15 | 100.00 | | |
| | | | | -Asymptotic | |
| | | | | Normal- | |
| | | ROC | Std. | [95% Conf. | |
| | Obs | Area | Err. | Interval] | |
| elafin_val | 12 | 0.9429 | 0.0680 | 0.80955 | 1.00000 |
| elafin_cat | 12 | 0.7857 | 0.1010 | 0.58773 | 0.98370 |
| Ho: area (elafin_val) = | | | | | |
| area (elafin_cat) | | | | | |
| chi 2 (1) = 2.63 | | | | | |
| Prob > chi 2 = 0.1048 | | | | | |

Results for gestation of 16 (2-week period, from 15 to 16+6).

Key
frequency
row percentage

| | del_34 | | | | |
|---|---|---|---|---|---|
| elafin_cat | 0 | 1 | Total | | |
| <50 ng/ml | 9 | 1 | 10 | | |
| | 90.00 | 10.00 | 100.00 | | |
| 50-100 | 5 | 2 | 7 | | |
| ng/ml | 71.43 | 28.57 | 100.00 | | |
| 100-200 | 8 | 5 | 13 | | |
| ng/ml | 61.54 | 38.46 | 100.00 | | |
| 200 | 10 | 6 | 16 | | |
| ng/ml+ | 62.50 | 37.50 | 100.00 | | |
| Total | 32 | 14 | 46 | | |
| | 69.57 | 30.43 | 100.00 | | |
| | | | | -Asymptotic | |
| | | | | Normal- | |
| | | ROC | Std. | [95% Conf. | |
| | Obs | Area | Err. | Interval] | |
| elafin_val | 45 | 0.6613 | 0.0870 | 0.49082 | 1.83176 |
| elafin_cat | 45 | 0.6371 | 0.0824 | 0.47556 | 0.79863 |
| Ho: area (elafin_val) = | | | | | |
| area (elafin_cat) | | | | | |
| chi 2 (1) = 0.58 | | | | | |
| Prob > chi 2 = 0.4451 | | | | | |

Key
frequency
row percentage

| | del_37 | | | | |
|---|---|---|---|---|---|
| elafin_cat | 0 | 1 | Total | | |
| <50 ng/ml | 8 | 2 | 10 | | |
| | 80.00 | 20.00 | 100.00 | | |
| 50-100 | 5 | 2 | 7 | | |
| ng/ml | 71.43 | 28.57 | 100.00 | | |
| 100-200 | 8 | 5 | 13 | | |
| ng/ml | 61.54 | 38.46 | 100.00 | | |
| 200 | 7 | 9 | 16 | | |
| ng/ml+ | 43.75 | 56.25 | 100.00 | | |
| Total | 28 | 18 | 46 | | |
| | 60.87 | 39.13 | 100.00 | | |
| | | | | -Asymptotic | |
| | | | | Normal- | |
| | | ROC | Std. | [95% Conf. | |
| | Obs | Area | Err. | Interval] | |
| elafin_val | 45 | 0.6723 | 0.0833 | 0.50896 | 0.83557 |
| elafin_cat | 45 | 0.6513 | 0.0817 | 0.49119 | 0.81133 |
| Ho: area (elafin_val) = | | | | | |
| area (elafin_cat) | | | | | |
| chi 2 (1) = 0.53 | | | | | |
| Prob > chi 2 = 0.4647 | | | | | |

Results for gestation of 18 (2-week period, from 17 to 18+6).

Key
frequency
row percentage

| | del_34 | | | | |
|---|---|---|---|---|---|
| elafin_cat | 0 | 1 | Total | | |
| <50 ng/ml | 11 | 2 | 13 | | |
| | 84.62 | 15.38 | 100.00 | | |
| 50-100 | 10 | 2 | 12 | | |
| ng/ml | 83.33 | 16.67 | 100.00 | | |
| 100-200 | 10 | 4 | 14 | | |
| ng/ml | 71.43 | 28.57 | 100.00 | | |
| 200 | 9 | 5 | 14 | | |
| ng/ml+ | 64.29 | 35.71 | 100.00 | | |
| Total | 40 | 13 | 53 | | |
| | 75.47 | 24.53 | 100.00 | | |
| | | | | -Asymptotic | |
| | | | | Normal- | |
| | | ROC | Std. | [95% Conf. | |
| | Obs | Area | Err. | Interval] | |
| elafin_val | 51 | 0.6275 | 0.0949 | 0.44157 | 0.81349 |
| elafin_cat | 51 | 0.6275 | 0.0898 | 0.45159 | 0.80348 |
| Ho: area (elafin_val) = | | | | | |
| area (elafin_cat) | | | | | |
| chi 2 (1) = 0.00 | | | | | |
| Prob > chi 2 = 1.0000 | | | | | |

Key
frequency
row percentage

| | del_37 | | | | |
|---|---|---|---|---|---|
| elafin_cat | 0 | 1 | Total | | |
| <50 ng/ml | 11 | 2 | 13 | | |
| | 84.62 | 15.38 | 100.00 | | |
| 50-100 | 10 | 2 | 12 | | |
| ng/ml | 83.33 | 16.67 | 100.00 | | |
| 100-200 | 9 | 5 | 14 | | |
| ng/ml | 64.29 | 35.71 | 100.00 | | |
| 200 | 6 | 8 | 14 | | |
| ng/ml+ | 42.86 | 57.14 | 100.00 | | |
| Total | 36 | 17 | 53 | | |
| | 67.92 | 32.08 | 100.00 | | |
| | | | | -Asymptotic | |
| | | | | Normal- | |
| | | ROC | Std. | [95% Conf. | |
| | Obs | Area | Err. | Interval] | |
| elafin_val | 51 | 0.7422 | 0.0813 | 0.58296 | 0.90147 |
| elafin_cat | 51 | 0.7171 | 0.0768 | 0.56657 | 0.86769 |
| Ho: area (elafin_val) = | | | | | |
| area (elafin_cat) | | | | | |
| chi 2 (1) = 1.16 | | | | | |
| Prob > chi 2 = 0.2807 | | | | | |

Results for gestation of 20 (2-week period, from 19 to 20+6).

Key
frequency
row percentage

| | del_34 | | | | |
|---|---|---|---|---|---|
| elafin_cat | 0 | 1 | Total | | |
| <50 ng/ml | 12 | 1 | 13 | | |
| | 92.31 | 7.69 | 100.00 | | |
| 50-100 | 5 | 3 | 8 | | |
| ng/ml | 62.50 | 37.50 | 100.00 | | |
| 100-200 | 13 | 1 | 14 | | |
| ng/ml | 92.86 | 7.14 | 100.00 | | |
| 200 | 13 | 6 | 19 | | |
| ng/ml+ | 68.42 | 31.58 | 100.00 | | |
| Total | 43 | 11 | 54 | | |
| | 79.63 | 20.37 | 100.00 | | |
| | | | | -Asymptotic | |
| | | | | Normal- | |
| | | ROC | Std. | [95% Conf. | |
| | Obs | Area | Err. | Interval] | |
| elafin_val | 52 | 0.6120 | 0.0955 | 0.42474 | 0.79921 |
| elafin_cat | 52 | 0.6341 | 0.0947 | 0.44850 | 0.81979 |
| Ho: area (elafin_val) = | | | | | |
| area (elafin_cat) | | | | | |
| chi 2 (1) = 0.71 | | | | | |
| Prob > chi 2 = 0.3979 | | | | | |

Key
frequency
row percentage

| | del_37 | | | | |
|---|---|---|---|---|---|
| elafin_cat | 0 | 1 | Total | | |
| <50 ng/ml | 11 | 2 | 13 | | |
| | 84.62 | 15.38 | 100.00 | | |
| 50-100 | 5 | 3 | 8 | | |
| ng/ml | 62.50 | 37.50 | 100.00 | | |
| 100-200 | 9 | 5 | 14 | | |
| ng/ml | 64.29 | 35.71 | 100.00 | | |
| 200 | 12 | 7 | 19 | | |
| ng/ml+ | 63.16 | 36.84 | 100.00 | | |
| Total | 37 | 17 | 54 | | |
| | 68.52 | 31.48 | 100.00 | | |
| | | | | -Asymptotic | |
| | | | | Normal- | |
| | | ROC | Std. | [95% Conf. | |
| | Obs | Area | Err. | Interval] | |
| elafin_val | 52 | 0.6151 | 0.0817 | 0.45492 | 0.77534 |
| elafin_cat | 52 | 0.6118 | 0.0787 | 0.45752 | 0.76601 |
| Ho: area (elafin_val) = | | | | | |
| area (elafin_cat) | | | | | |
| chi 2 (1) = 0.02 | | | | | |
| Prob > chi 2 = 0.8944 | | | | | |

### Example 4

To compare elafin expression in cases and controls prior to randomised treatment, samples up to and including the visit at which the cervix shortened, but prior to randomised treatment over a period of time from 16 to 24 weeks' were included and the average difference between cases and controls determined using a correction for effect of gestation (2-weekly categories). Results were expressed as ratios of elafin concentration in cases and controls with 95% confidence intervals. A p value of less than 0.05 was considered to indicate statistical significance. Actual p-values are given (usually to 2 decimal places), except for very small values, shown as p<0.001.

### Results

Concentrations of elafin were higher in the CVF of the short cervix group compared to controls, regardless of gestation and treatment (ratio 2.71, CI 1.94 to 3.79, p<0.0005) —see FIG. 3.

Elafin concentrations remained high at the gestations when cervical shortening was first detected (ratio 3.03 CI 1.92-4.81, p<0.0005). Elafin concentrations were unaltered by treatment (insertion of a cerclage or vaginal progesterone daily; ratio 1.28, CI: 0.88-1.87, p=0.196). Raised elafin concentrations <24 weeks' were associated with SPTB <37 weeks (ratio 1.79, CI: 1.05-3.05, p=0.034).

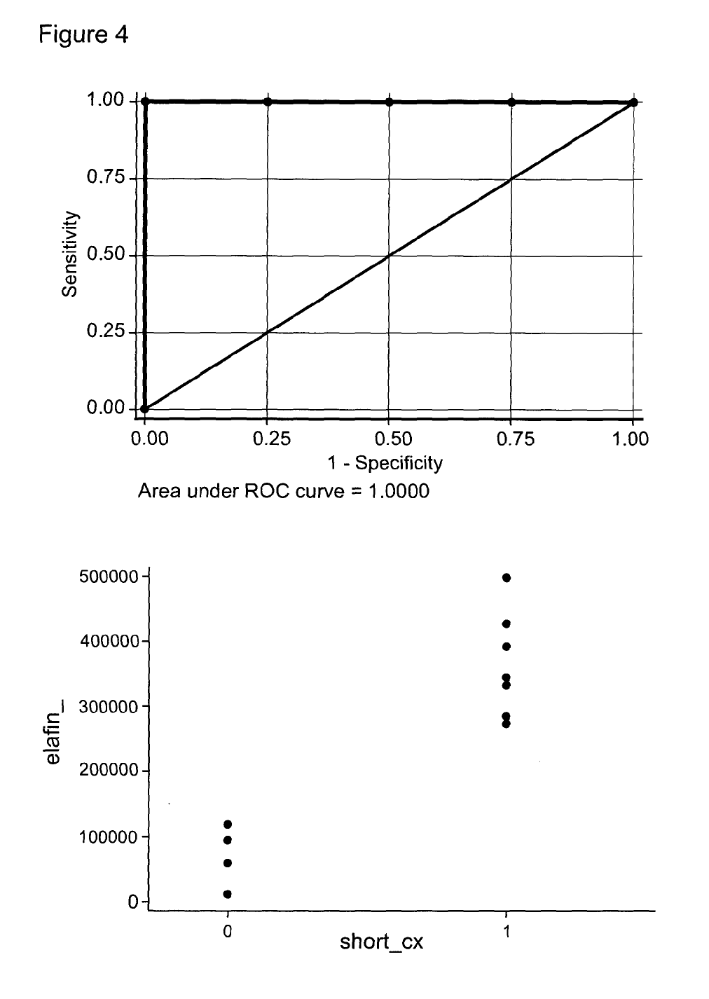

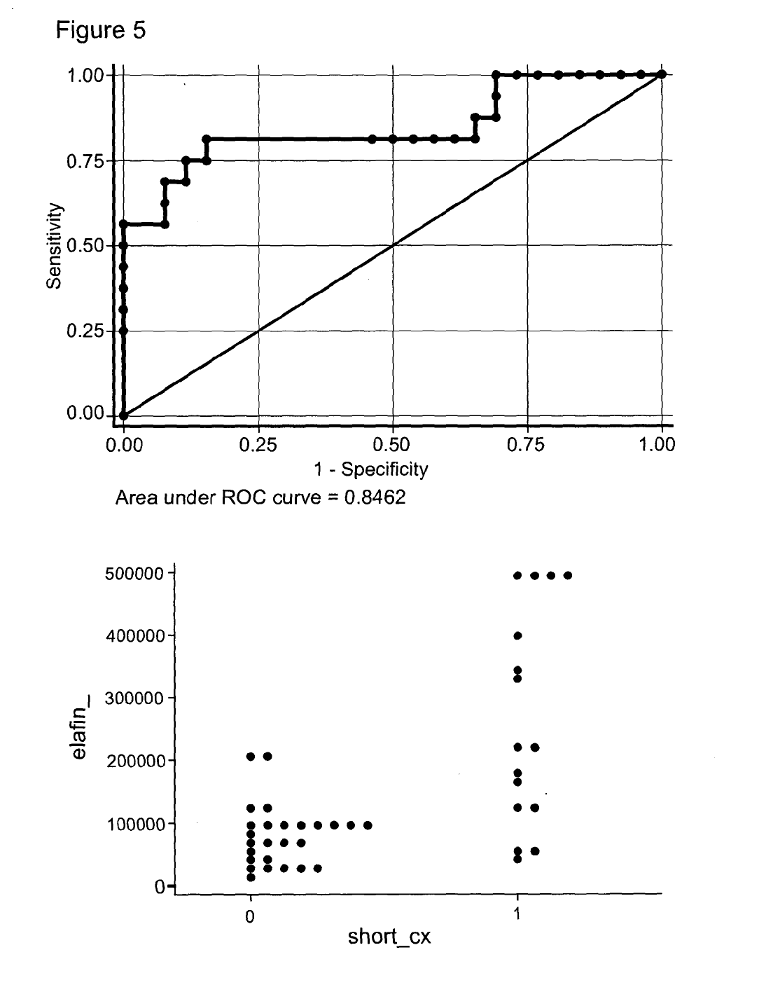

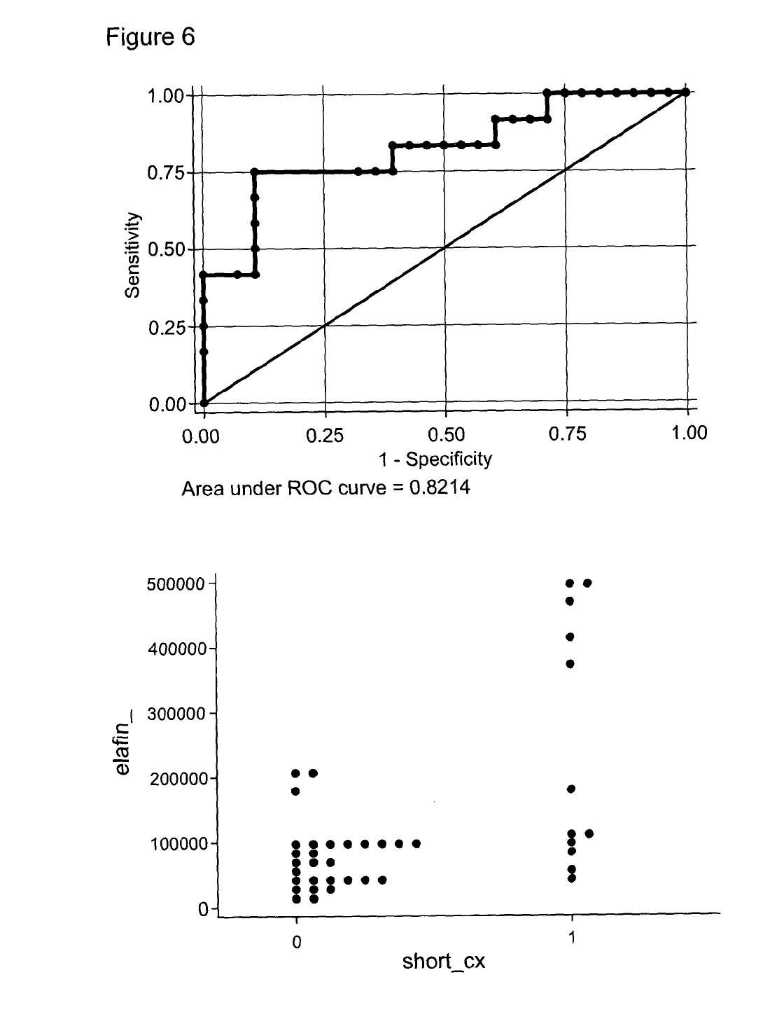

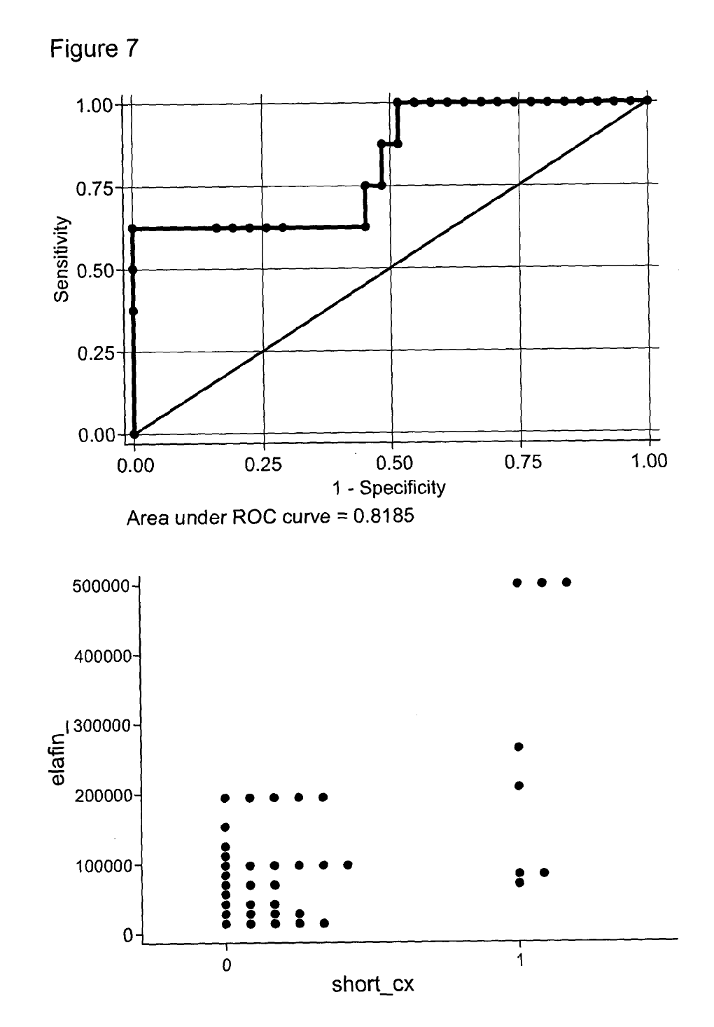

Elafin concentrations predicted cervical shortening from 14-20 weeks' in cases. Data are shown in FIGS. 4-7.

The majority of women recruited to the study were of black ethnicity. We analyzed the data to assess the interactions between elafin concentrations, ethnicity and prediction of a short cervix. Elafin predicted a short cervix in both black and white women and elafin was as stronger predictor than ethnicity.

Serum 25-Hydroxy Vitamin D concentrations were unrelated to cervico-vaginal elafin concentrations. However, the majority of women were women in the study were 25-Hydroxy Vitamin D deficient (<25 nmol/L) or insufficient (>25 ng/ml but <75 ng/ml).

TABLE
Serum 25-Hydroxy Vitamin D status
of women include in the Flafin study.
Ethnicity

| | | | | | Total |
|---|---|---|---|---|---|
| | | | | | % |
| | White | Black | Asian | Other | (n |
| | % | % | % | % | women |
| nmol/ | (n | (n | (n | (n | all |
| L | women) | women) | women) | women) | groups) |
| <25 | 20 | 20.83 | 100 | 0 | 20.90 |
| | (3) | (10) | (1) | (0) | (14) |
| 25-49.9 | 40 | 70.83 | 0 | 100 | 64.18 |
| | (6) | (34) | (0) | (3) | (43) |
| 50-74.9 | 26.67 | 6.25 | 0 | 0 | 10.45 |
| | (4) | (3) | (0) | (0) | (7) |
| >=75 | 13.33 | 2.08 | 0 | 0 | 4.48 |
| | (2) | (1) | (0) | (0) | (3) |
| Total | 100 | 100 | 100 | 100 | 100 |
| | (15) | (48) | (1) | (3) | (67) |

### Example 5

### Relationship Between Cervical-Vaginal Fluid Elafin Concentrations and Subsequent Cervical Shortening in Women at High Risk of Spontaneous Preterm Birth

Introduction:

Elafin (SKALP), a natural antimicrobial peptide with antibacterial/antiprotease properties, is an important component of the innate immune system. It is hypothesised that elafin production in cervico-vaginal fluid (CVF) will be altered in women at risk of spontaneous preterm birth (STPB) associated with inflammation/infection.

Aim:

The aim of this study was to determine the relationship between CVF elafin concentrations and cervical length in a cohort of woman at high risk of SPTB.

Methods:

Elafin concentrations were measured in 437 CVF samples (taken at two-weekly intervals between 14-28 weeks') from 74 pregnant women recruited as part of a prospective longitudinal study of inflammation and cervical length (CLIC study). All women were asymptomatic but high risk for SPTB; controls (n=38, who did not develop a short cervix) and women who developed a short cervix <25 mm (n=36) who were randomised to either cerclage or vaginal progesterone (400 mg/od). Elafin was measured by ELISA. Logged data were analysed using random-effects interval regression (Stata), with 28 low censored sample values and 85 high values.

Results:

Mean concentrations of elafin were consistently higher in the CVF of the short cervix group, regardless of gestation and treatment (ratio 2.71, CI 1.94 to 3.79, p<0.0005). Elafin concentrations >200,000 pg/ml predicted cervical shortening from 14 weeks (n=11, ROC area=1.00, p=0.0082), remained high when cervical shortening was first detected (ratio 3.03 CI 1.92-4.81, p<0.0005) and was consistently raised across gestation. Elafin concentrations were unaltered by treatment with cerclage or progesterone (ratio 1.28, CI: 0.88-1.87, p=0.196). Raised elafin concentrations before 24 weeks' were associated with SPTB <37 weeks (ratio 1.79, CI: 1.05-3.05, p=0.034).

Conclusion:

This novel prospective study of high-risk asymptomatic women demonstrates that CVF elafin concentrations may be useful for the early predication of SPTB prior to cervical shortening. Raised elafin concentrations may reflect a reactive response to the presence of infection rather than being a causative factor. The utility of elafin to predict SPTB and target therapies can be investigated in a larger cohort.

### Example 6

### Cervical-Vaginal Fluid Elafin Concentrations and Subsequent Cervical Shortening in Women at High Risk of Spontaneous Preterm Birth

Introduction:

Elafin, a natural antimicrobial peptide, is an important component of the innate immune system. It is hypothesised that elafin production in cervico-vaginal fluid (CVF) will be altered in women at risk of spontaneous preterm birth (STPB) associated with inflammation/infection.

Aim:

To determine the relationship between CVF elafin concentrations and cervical length in women at high risk of SPTB.

Methods:

Elafin concentrations were measured in 437 CVF samples (taken two-weekly between 14-28 weeks') from 74 pregnant women (asymptomatic, high risk for SPTB).

Controls:

women who did not develop a short cervix (n=38); cases: women who developed a short cervix <25 mm, n=36) and were randomised to either cerclage or vaginal progesterone (400 mg/od). Elafin was measured by ELISA. Logged data were analysed using random-effects interval regression.

Results:

Concentrations of elafin were higher in the CVF of the short cervix group, regardless of gestation and treatment (ratio 2.71, CI 1.94 to 3.79, p<0.0005). Elafin concentrations predicted cervical shortening from 14 weeks' (n=11, ROC area=1.00, p=0.0082), and remained high when cervical shortening was first detected (ratio 3.03 CI 1.92-4.81, p<0.0005). Elafin concentrations were unaltered by cerclage or progesterone (ratio 1.28, CI: 0.88-1.87, p=0.196). Raised elafin concentrations <24 weeks' were associated with SPTB <37 weeks (ratio 1.79, CI: 1.05-3.05, p=0.034).

Conclusion:

These data show that CVF elafin concentrations may be useful for the early predication of SPTB prior to cervical shortening.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of determining a risk of preterm labour in a subject, the method comprising;
a) providing a sample from said subject, wherein said sample comprises one or more of cervicovaginal fluid, endocervical fluid, foetal membrane fluid, amniotic fluid, serum or plasma;
b) measuring the elafin concentration in said sample; and
c) comparing the elafin concentration of (b) to a reference standard;
wherein if the elafin concentration of (b) is elevated compared to the reference standard, then it is indicated that said subject has an increased likelihood of preterm labour.

2. The method according to claim 1 wherein the sample comprises cervicovaginal fluid.

3. The method according to claim 1, wherein the sample is diluted to achieve an elafin concentration in the range 878 to 10,000 picograms per millilitre (pg/ml) before measurement of elafin concentration.

4. The method according to claim 1, wherein the elafin concentration is determined by ELISA.

5. The method according to claim 1 wherein said sample was obtained from said subject at 24 weeks or less of gestation.

6. The method according to claim 5, wherein said sample was obtained at 14-20 weeks of gestation.

7. The method according to claim 6, wherein said sample was obtained at 14, 16, 18 or 20 weeks of gestation.

8. The method according to claim 7, wherein said sample was obtained at 14 weeks of gestation.

9. A method of treating a subject at risk of preterm labour, said method comprising performing the method according to claim 1, wherein if the subject is found to have an increased likelihood of preterm labour then cervical cerclage or vaginal progesterone is administered to said subject.

10. The method according to claim 9, wherein the subject is at high risk of preterm labour.

11. The method according to claim 9, wherein the subject is at low risk of preterm labour.

12. The method according to claim 9, wherein the subject has bulging membranes.

13. A method of determining the risk of preterm labour in a subject, the method comprising; a) performing the method according to claim 9; b) measuring the foetal fibronectin level in the sample; c) comparing the level of fibronectin determined with the level of fibronectin in a reference sample; and d) if the subject has elevated elafin relative to the reference sample and has elevated fibronectin relative to the reference sample, then it is indicated that said subject has an increased likelihood of preterm labour.

14. A method of determining a risk of preterm labour in a subject, the method comprising:
a) providing a first sample from said subject, wherein said first sample comprises one or more of cervicovaginal fluid, endocervical fluid, foetal membrane fluid, amniotic fluid, serum or plasma; wherein said first sample was obtained at a first stage of gestation,
b) measuring the elafin concentration in said first sample;
c) providing a second sample from said subject, wherein said second sample comprises one or more of cervicovaginal fluid, endocervical fluid, foetal membrane fluid, amniotic fluid, serum or plasma; wherein said second sample was obtained at a second stage of gestation, said second stage of gestation being later than said first stage of gestation,
d) measuring the elafin concentration in said second sample; and
e) comparing the elafin concentration of (d) to that in (b),
wherein if the elafin concentration of (d) is elevated compared to that in (b), then it is determined that that said subject has an increased likelihood of preterm labour.

15. A method of aiding the diagnosis of a short cervix in a subject, the method comprising;
a) providing a sample from said subject, wherein said sample comprises one or more of cervicovaginal fluid, endocervical fluid, foetal membrane fluid, amniotic fluid, serum or plasma;
b) measuring the elafin concentration in said sample; and
c) comparing the elafin concentration of (b) to a reference standard;
wherein if the elafin concentration of (b) is elevated compared to the reference standard then it is indicated that said subject has an increased likelihood of a short cervix.

16. A method of determining a risk of preterm labour in a subject, the method comprising performing the method according to claim 15, wherein if the subject is determined to have an increased risk of a short cervix, then the subject is scanned periodically thereafter to monitor cervical length, wherein a short cervix indicates an increased likelihood of preterm labour.

17. A method of determining a risk of preterm labour in a subject, the method comprising;
a) providing a sample from said subject, wherein said sample comprises one or more of cervicovaginal fluid, endocervical fluid, foetal membrane fluid, amniotic fluid, serum or plasma; and
b) measuring the elafin concentration in said sample;
wherein if the elafin concentration of (b) is 200 ng/ml or more then it is indicated that said subject has an increased likelihood of preterm labour.

18. A method of aiding the diagnosis of a short cervix in a subject, the method comprising;
a) providing a sample from said subject, wherein said sample comprises one or more of cervicovaginal fluid, endocervical fluid, foetal membrane fluid, amniotic fluid, serum or plasma; and
b) measuring the elafin concentration in said sample;
wherein if the elafin concentration of (b) is 200 ng/ml or more then it is indicated that said subject has an increased likelihood of a short cervix.

19. The method according to claim 17, wherein the sample comprises cervicovaginal fluid.

20. The method according to claim 17, wherein the sample is diluted to achieve an elafin concentration in the range 878 to 10,000 picograms per millilitre (pg/ml) before measurement of elafin concentration.

21. The method according to claim 17, wherein said sample was obtained at 14 weeks of gestation.

22. A diagnostic kit for assessing risk of preterm labour, comprising:
quantifiable ligands for elafin and for fibronectin.

23-24. (canceled)
